(19) United States
(12) Patent Application Publication
(10) Pub. No.: US 2025/0312146 A1
(43) Pub. Date: Oct. 9, 2025

(54) DEVICES AND METHODS FOR LENS MANAGEMENT

(71) Applicant: Johnson & Johnson Surgical Vision, Inc., Irvine, CA (US)

(72) Inventors: David RUDDOCKS, Celina, TX (US); Vanessa CORRALES-YANG, Mission Viejo, CA (US); Joseph PAZ, San Marcos, CA (US); Michael LUM, Lake Forest, CA (US); Jason SAN JOSE, Foothill Ranch, CA (US); Mark COLE, Trabuco Canyon, CA (US); David ISRAEL, Thousand Oaks, CA (US); Edward GERAGHTY, Rancho Santa Margarita, CA (US)

(21) Appl. No.: 19/047,557

(22) Filed: Feb. 6, 2025

Related U.S. Application Data
(60) Provisional application No. 63/575,553, filed on Apr. 5, 2024.
(60) Provisional application No. 63/708,491, filed on Oct. 17, 2024.

(51) Int. Cl. A61F 2/16

(52) U.S. Cl. CPC A61F 2/1678

(57) ABSTRACT

An example lens management device can include a sleeve, a modular cartridge configured to house a lens, and a tray configured to transition from a first position to a second position. The tray can include a first tray component configured to removably hold the modular cartridge, and a second tray component movably attached to the first tray component. The modular cartridge can be configured to house the lens in a third position when the tray is in the first position, and in a fourth position when the tray is in the second position, the fourth position being distal to the third position. Transitioning the tray from the first to the second position causes the sleeve to enter into the modular cartridge such that the distal tip of the sleeve is positioned to engage with the lens and move the lens from the third position to the fourth position.

### RELATED APPLICATIONS

The present application claims priority to U.S. Provisional Application No. 63/708,461, filed Oct. 17, 2024, and U.S. Provisional Application No. 63/575,553, filed Apr. 5, 2024, the entire contents of each of which are fully incorporated herein by reference.

### FIELD

The present invention generally relates to devices and methods for lens management and delivery. More specifically, certain embodiments relate to devices and methods for inserting an intraocular lens into a patient's eye.

### BACKGROUND

Devices and methods for managing and delivering an implantable lens, such as an intraocular lens (IOL), are typically configured to apply the force required to advance the lens through a delivery device and into an eye of a patient. Implantable lenses are typically configured with different types of haptics that help hold the lens in place once inserted in the patient's eye. These haptics, along with the central optic of the lens, must be manipulated precisely during management and delivery to ensure the lens is situated properly in the patient's eye, such as during cataract surgery.

Accordingly, alternative devices and methods for lens management and delivery would be useful.

### SUMMARY

The present invention is directed to devices and methods for lens delivery management.

An example lens management device is provided. The lens management device can include a modular cartridge configured to house a lens, and a tray configured to transition from a first position to a second position. The tray can include a first tray component configured to removably hold the modular cartridge, and a second tray component movably attached to the first tray component. The lens management device can further include a sleeve, having a distal tip and a center lumen, configured to move distally into the modular cartridge as the tray transitions from the first position to the second position. The modular cartridge can be configured to house the lens in a third position when the tray is in the first position, and in a fourth position when the tray is in the second position, the fourth position being distal to the third position. Transitioning the tray from the first position to the second position causes the sleeve to enter into the modular cartridge such that the distal tip of the sleeve is positioned to engage with the lens and move the lens from the third position to the fourth position.

Another example lens management device is provided. The lens management device can include a tray configured to transition from a first position to a second position. The tray can include a first tray component configured to hold a lens, and a second tray component movably attached to the first tray component. The lens management device can further include a sleeve, having a distal tip and a center lumen, configured to move distally into the first tray component as the tray transitions from the first position to the second position. Transitioning the tray from the first position to the second position moves the sleeve into the first tray component such that the distal tip of the sleeve is positioned to engage with the lens and move the lens from a third position within the first tray component to a fourth position within the first tray component, the fourth position being distal to the third position.

A method for lens management is provided. The method can include providing a lens management device that has a modular cartridge configured to house the lens, a tray configured to removably engage with a sleeve and the modular cartridge, and the sleeve having a distal tip and a center lumen. The tray can include a first tray component configured to removably hold the modular cartridge, and a second tray component movably attached to the first tray component. The method can further include moving the tray from a first position to a second position thereby moving the sleeve into the modular cartridge such that the distal tip of the sleeve engages with the lens and moves the lens from a third position within the modular cartridge to a fourth position within the modular cartridge, the fourth position being distal to the third position.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 is an illustration of an example lens management device according to aspects of the present invention.

FIG. 2 is an illustration of an example lens management device according to aspects of the present invention.

FIG. 3 is an illustration of an example lens management device according to aspects of the present invention.

FIG. 4A is a top perspective view of an example first tray component used in a lens management device according to aspects of the present invention.

FIG. 4B is a bottom perspective view of the example first tray component of FIG. 4A according to aspects of the present invention.

FIG. 5 is an illustration of an example second tray component used in a lens management device according to aspects of the present invention.

FIG. 6A is an illustration of an example sleeve used in a lens management device according to aspects of the present invention.

FIG. 6B is an illustration of an example distal tip of a sleeve used in a lens management device according to aspects of the present invention.

FIG. 6C is an illustration of an example distal tip of a sleeve used in a lens management device according to aspects of the present invention.

FIG. 7 is an illustration of an example modular cartridge used in a lens management device according to aspects of the present invention.

FIG. 8A is a top perspective view of an example lens module used in a lens management device according to aspects of the present invention.

FIG. 8B is a bottom perspective view of the example lens module of FIG. 8A according to aspects of the present invention.

FIG. 9 is an illustration of an example lens module used in a lens management device according to aspects of the present invention.

FIG. 10 is an illustration of an example cartridge used in a lens management device according to aspects of the present invention.

FIG. 11 is an illustration of an example modular cartridge used in a lens management device according to aspects of the present invention.

FIG. 12 is a top view of example components used in a lens management device according to aspects of the present invention.

FIG. 13A is a cross-sectional view of the modular cartridge and sleeve with the lens in a first lens position used in a lens management device according to aspects of the present invention.

FIG. 13B is a cross-sectional view of the modular cartridge and sleeve with the lens in a second lens position used in a lens management device according to aspects of the present invention.

FIG. 14A is an illustration of an example lens delivery device according to aspects of the present invention.

FIG. 14B is an illustration of a push rod of a lens delivery device extending through the sleeve of the lens management device according to aspects of the present invention.

FIG. 14C is an illustration of a push rod of a lens delivery device extending through the sleeve of the lens management device according to aspects of the present invention.

FIG. 15 is a flowchart of an exemplary method for lens management according to aspects of the present invention.

### DETAILED DESCRIPTION

The example devices and methods described herein generally involve providing management of an implantable lens, such as an intraocular lens (IOL), by applying force that is required to fold a lens and deliver the lens into an eye of a patient. The disclosed example devices and methods provide improvements in lens management and delivery by separating the steps of preparation and delivery of the lens. In a first step, the devices and methods disclosed herein provide for at least partially folding the lens while advancing the lens from a storage location to a holding position within a modular cartridge. Hydration of the lens can be conducted prior to attachment of the lens to a handpiece. In a second step, the devices and methods disclosed herein provide for further folding the lens while further advancing the lens from the holding position into the patient's eye. It should be understood that the example devices and methods described herein can be used with different types of implantable lenses, including lenses with different types of haptics, as well as different foldable implantable medical devices.

Further, without intending to be limiting, there are instances where it may be desirable to apply the force required to fold a lens and deliver the lens into an eye of a patient in two steps. This may be when the lens that is to be inserted is comparatively larger than the desired diameter of the insertion tip; where a lens is formed from a material that is comparatively less pliable than other materials; where a lens is formed from a material that may be damaged if the force was applied in one application; where a lens is formed from a material that may be damaged if the force was applied through contact by only one lens-contacting pushrod tip portion; where a lens would benefit from folding with one material and contact with a lens-contacting pushrod tip of a different material or where a larger lens is desired to be implanted (e.g., with a phakic IOL).

The example devices and methods disclosed herein provide for improvement in the folding of the lens as it moves through the stages of preparation and delivery, as discussed above. The disclosed devices and methods provide enough force to advance the lens through a lens management device and into the patient's eye without resulting in improper folding of the lens optic or haptics. Further, the disclosed devices and methods create improved stability during lens advancement to reduce variability in lens delivery.

Various example devices and methods are presented herein. Features from each example are combinable with other examples as understood by persons skilled in the pertinent art.

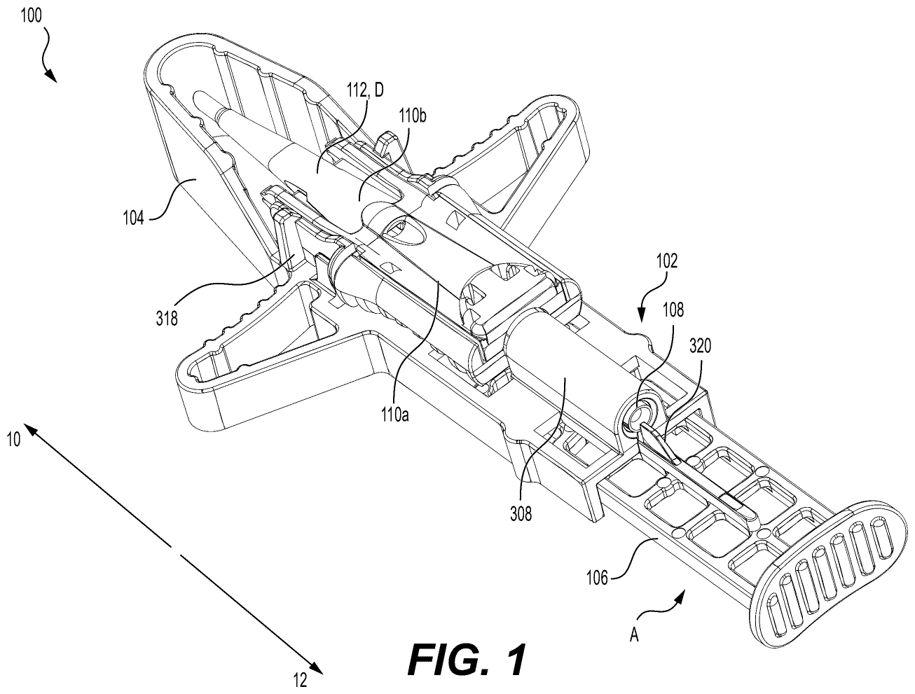

FIG. 1 is an illustration of an example lens management device 100. The device 100 includes a tray 102 that can include a first tray component 104 and a second tray component 106. In some embodiments, as further discussed herein, the second tray component 106 can be movable within the first tray component 104. For example, the second tray component 106 can be configured to slide distally 10 (e.g., in a telescoping fashion) into the first tray component 104. In other words, the tray 102 can start out in a first position A, and the first tray component 104 can remain stationary as the second tray component 106 is advanced into the first thereby transitioning the tray 102 from the first position A to a second position. The tray components 104, 106 can be plastic or may be formed from a rigid paper or cardboard or other recyclable material.

As discussed herein, a user, e.g., a physician, can push against the second tray component 106 to slide it into the first tray component 104 to apply the required force to advance an implantable lens 112, e.g., an IOL, to a position where it can undergo at least partial folding. This partial folding prepares the IOL to move further distally through the device 100 and into a patient's eye. As discussed herein, this advancement of the lens can take place in two separate steps or stages.

In some embodiments, the device 100 can further include a sleeve 108, as further discussed below. As a user pushes on the second tray component 106 to move it distally 10 into the first tray component 104, the sleeve 108 can also be advanced distally into the first tray component 104 to engage with the lens 112, as further discussed below.

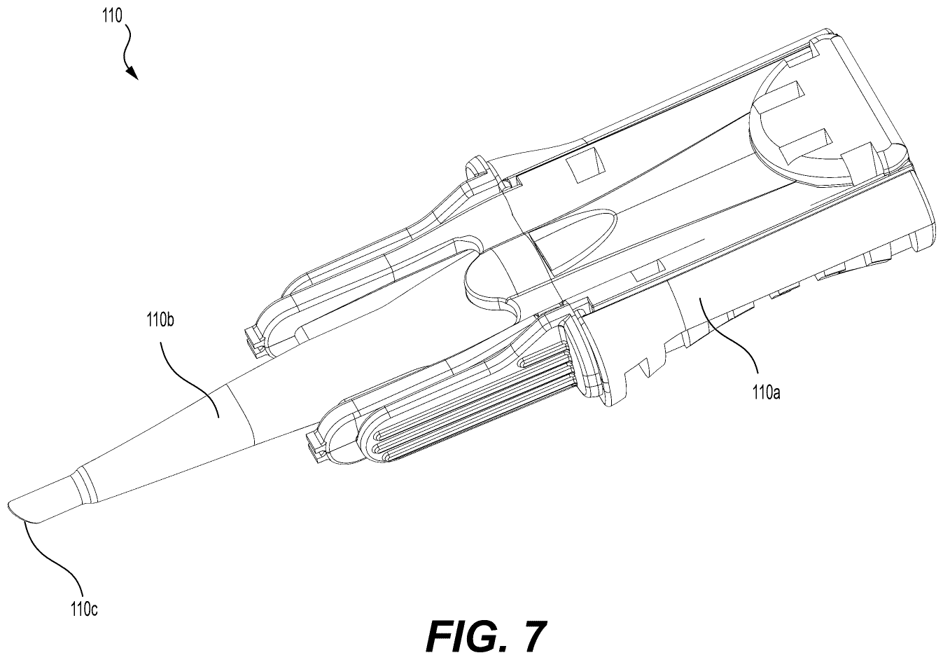

In some embodiments, the device 100 can further include a modular cartridge 110 (also shown in FIG. 7) configured to hold the lens 112, such as an IOL, in different positions throughout the delivery process, as further discussed below. The modular cartridge 110 can include a lens module 110a and a cartridge 110b that can be removably attached to one another, as further discussed below. In some embodiments, modular cartridge 110 may be a single cohesive piece. The modular cartridge 110 can be removably held in the first tray component 104 during shipment and storage of the device 100, as well as in the first stage of lens management and delivery, as further discussed herein. The modular cartridge 110 can be removably held by both a friction fit and an interference fit and/or where the friction fit and the interference fit are present at different portions of the modular cartridge 110.

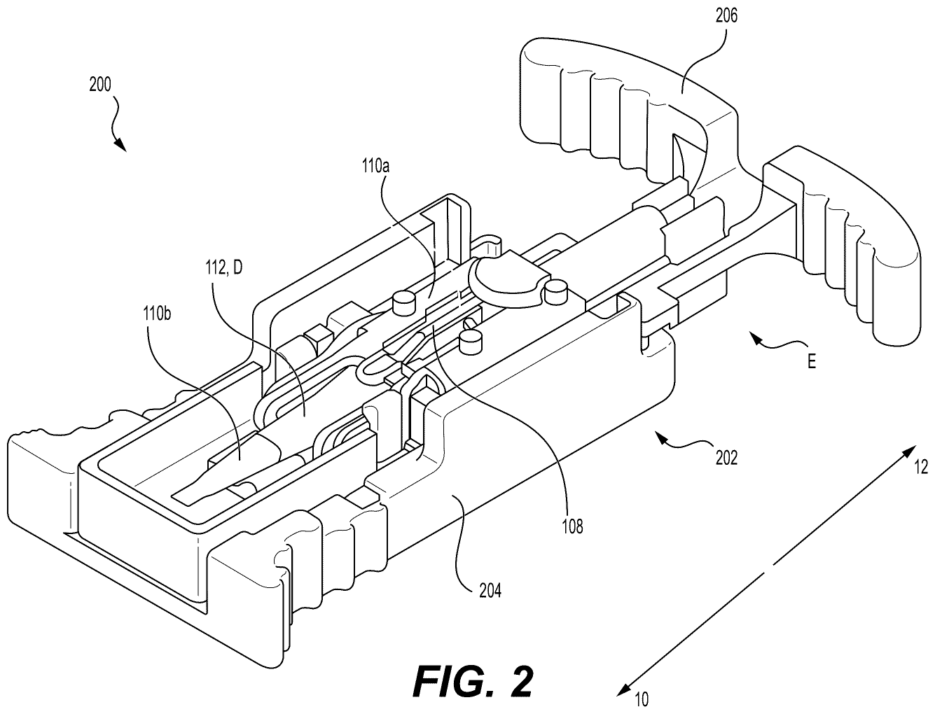

FIG. 2 is an illustration of another example lens management device 200 that may be similar to the lens management device 100 of FIG. 1 except in how the components of the tray engage with one another and thus move the sleeve to engage the lens. Lens management device 200 can include a tray 202 that can include a first tray component 204 and a second tray component 206 (e.g., finger pulls). The first and second tray components 204, 206 can be movable in relation to each other. In some embodiments, the first tray component 204 can be moved distally 10 in relation to second tray component 206, such as while the second tray component 206 is held stationary. In other embodiments, the second tray component 206 can be moved proximally 12 in relation to the first tray component 204, such as while the first tray component 204 is held stationary. In still other embodiments, the first tray component 204 can be moved distally 10 in relation to the second tray component 206 while the second tray component 206 is moved proximally 12 in relation to the first tray component 204. In other words, the tray 202 can start out in a first position E and can be transitioned to a second position by pulling the first and/or second tray components 204, 206 away from one another. For example, a user, e.g., a physician, can grab the first tray component 204 and the second tray component 206 and can pull the first tray component 204 and second tray component 206 away from each other to apply the required force to advance an implantable lens 112, e.g., an IOL, to a position where it can undergo at least partial folding. This partial folding prepares the IOL to move further distally through the device 200 and into a patient's eye. As discussed herein, this advancement of the lens can take place in two separate steps or stages.

In some embodiments, similar to device 100, the device 200 can include a modular cartridge 110 (e.g., including a lens module 110a and a cartridge 110b that can be removably attached to one another) configured to hold the lens 112, such as an IOL, in different positions throughout the delivery process, as further discussed below. The modular cartridge 110 can be removably held in the first tray component 204 during shipment and storage of the device 200, as well as in the first stage of lens management and delivery, as further discussed herein.

In some embodiments, similar to device 100, the device 200 can further include a sleeve 108, as further discussed herein. As a user pulls on the first and/or second tray components 204, 206, as discussed above, the sleeve 108 can also be advanced distally 10 into the first tray component 204 to engage with the lens 112, as further discussed below. That is, the motion of the second tray component 206 relative to the first tray component 204, as discussed above, slides the second tray component 206 proximally 12 and/or the first tray component 204 distally 10, and moves the modular cartridge 110 proximally 12 such that the sleeve 108 advances into the modular cartridge 110.

It should be understood that while the remaining figures in this disclosure are discussed with respect to first and second tray components 104, 106 of lens management device 100 (FIG. 1), first and second tray components 204, 206 of lens management device 200 (FIG. 2) can be substituted such that the lens management device can require either a pushing force (device 100) or a pulling force (device 200) to advance the sleeve 108 into the modular cartridge 110 such that the sleeve 108 engages with the lens 112, as discussed herein.

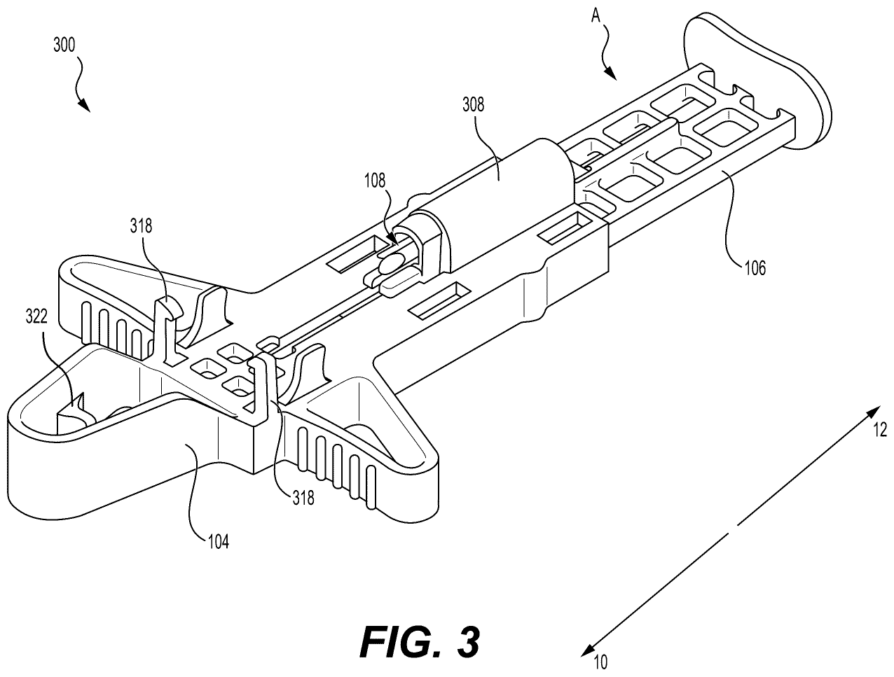

FIG. 3 is an illustration of an example lens management device 300. Device 300 may be similar to device 100 in FIG. 1 (or device 200 of FIG. 2), except that device 300 may not include the modular cartridge 110 or lens 112. For example, device 300 can include the first and second tray components 104, 106 (or 204, 206) as well as the sleeve 108. In some embodiments, the first tray component 104 can include a support 322 that can help prevent potential damage to the distal tip 110c of the cartridge 110 during removal, as discussed herein. For example, as the cartridge 110 is removed from the device (e.g., device 300), the distal tip 110c of the cartridge 110 may be inadvertently pushed downward, thereby colliding with a surface or object (e.g., the first tray component 104 or a tabletop surface) underneath the cartridge 110, and becoming damaged. Support 322 can be configured such that it is disposed underneath the distal tip 110c of the cartridge to prevent the distal tip 110c from being pushed or angled in a downward direction.

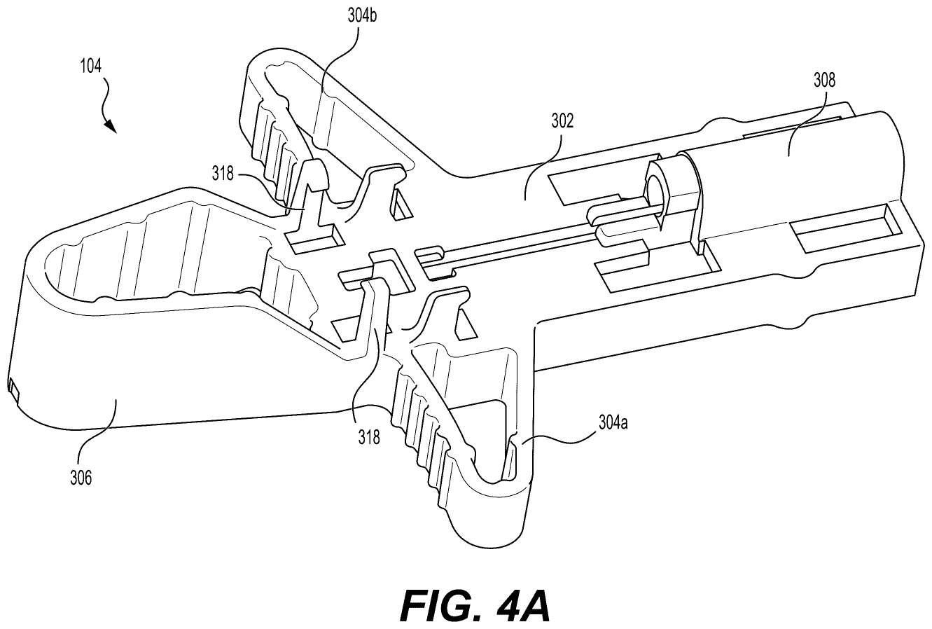

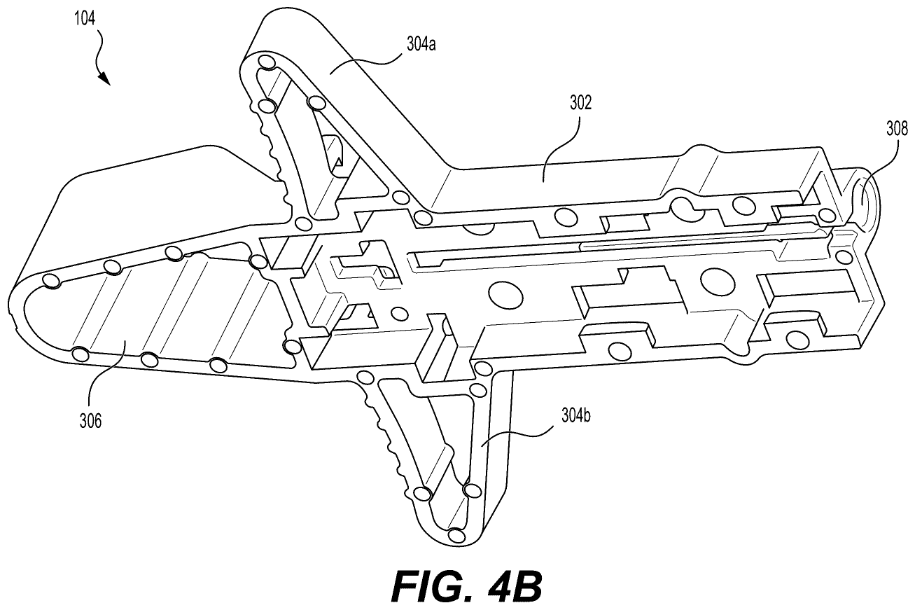

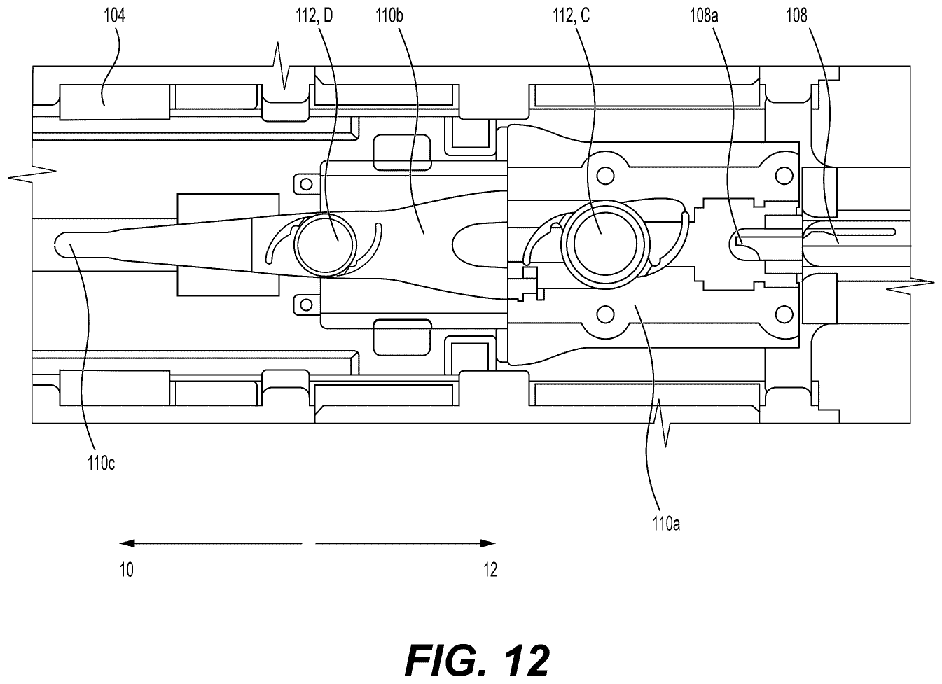

FIGS. 4A and 4B respectively provide top and bottom perspective views of an example first tray component 104 of a lens management device (e.g., 100). As shown, the first tray component 104 can include a body 302 configured in a shape to removably hold a modular cartridge 110 and lens 112 during shipment and storage, as well as a first stage of the delivery process. For example, as particularly shown in FIG. 12, the first tray component 104 can removably hold a modular cartridge 110 which itself can hold the lens 112 in a lens first position C. In this lens first position C, the lens 112 can be shipped and/or stored in the first tray component 104. Additionally, as further discussed below, in this lens first position C, the lens 112 can be hydrated to prepare it for delivery. Once the second component 106 is advanced or pushed distally 10 into the first tray component 104 (or the first and second tray components 204, 206 are pulled apart from one another) thereby advancing the sleeve 108 distally into the modular cartridge 110, the sleeve 108 can push the lens 112 distally within the modular cartridge 110 from its first lens position C to a second lens position D, as further discussed below.

Turning back to FIGS. 4A and 4B, in some embodiments, the first tray component 104 may further include one or more wings 304a, 304b, as well as a front protrusion 306 that a user, e.g., a physician, can use to hold onto the first tray component 104 during management of the lens. This front protrusion 306 can also serve to protect the distal end or tip 110c of the cartridge during shipping, storage and initial manipulation by the physician or their staff.

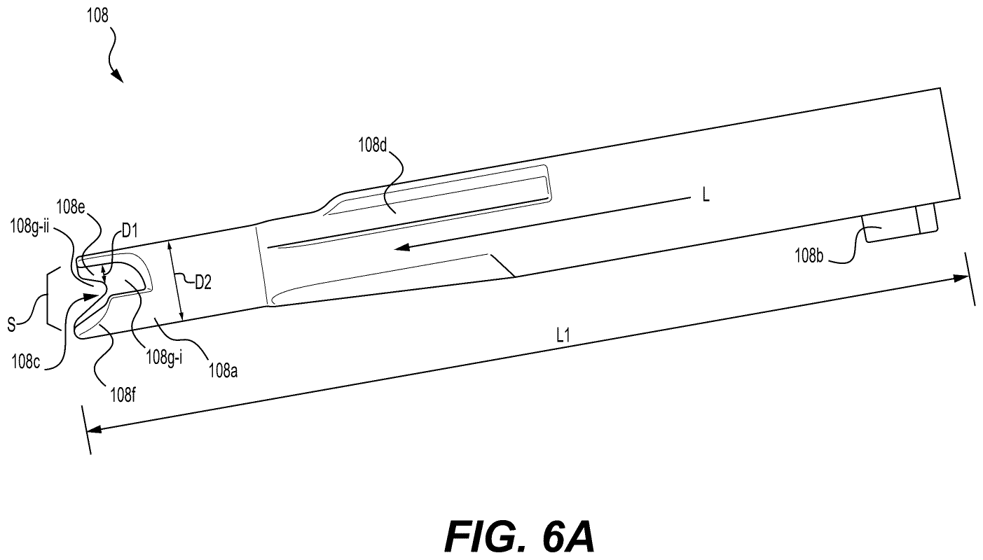

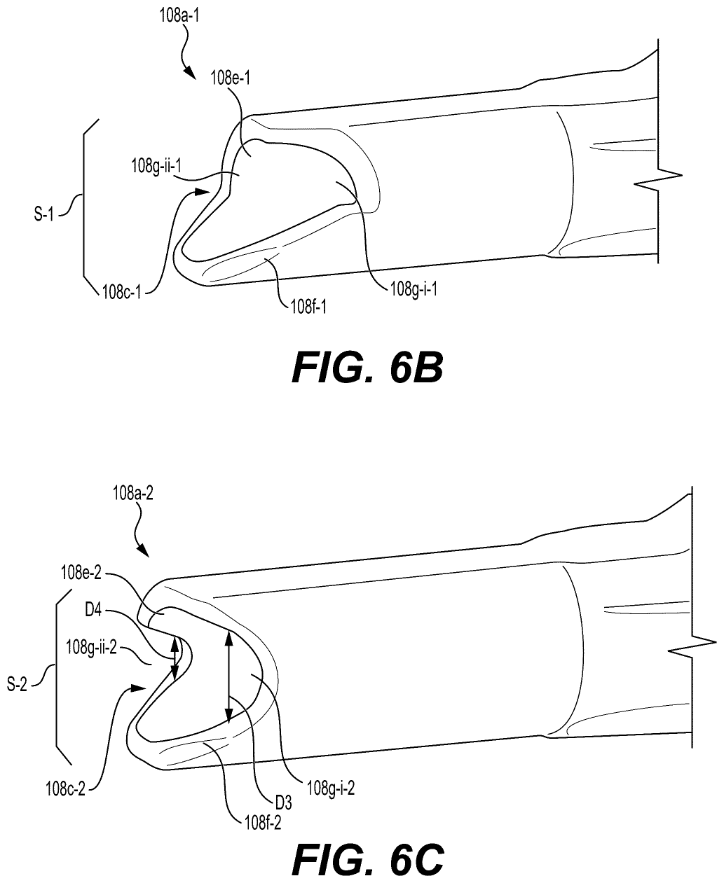

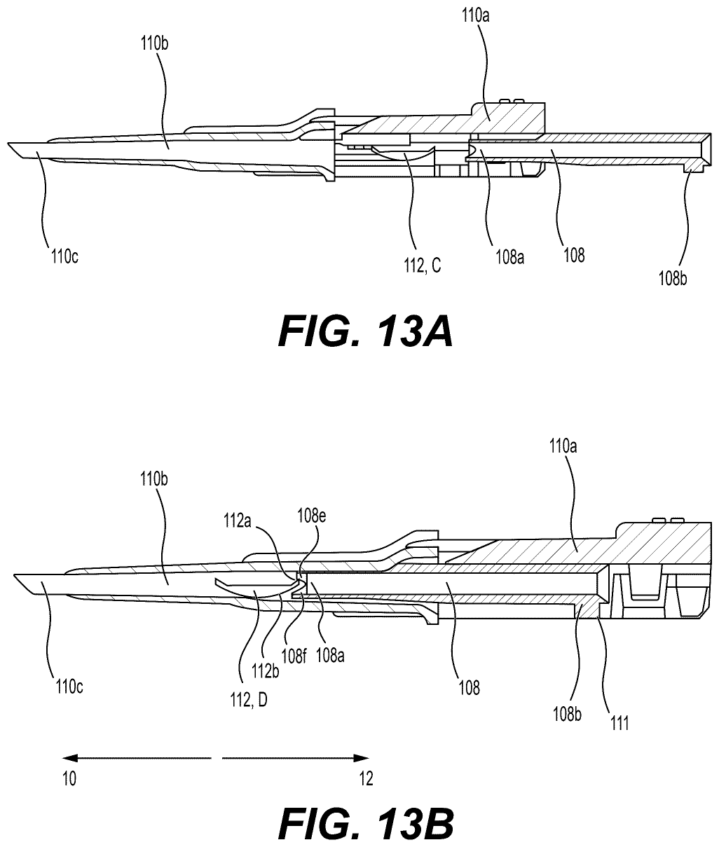

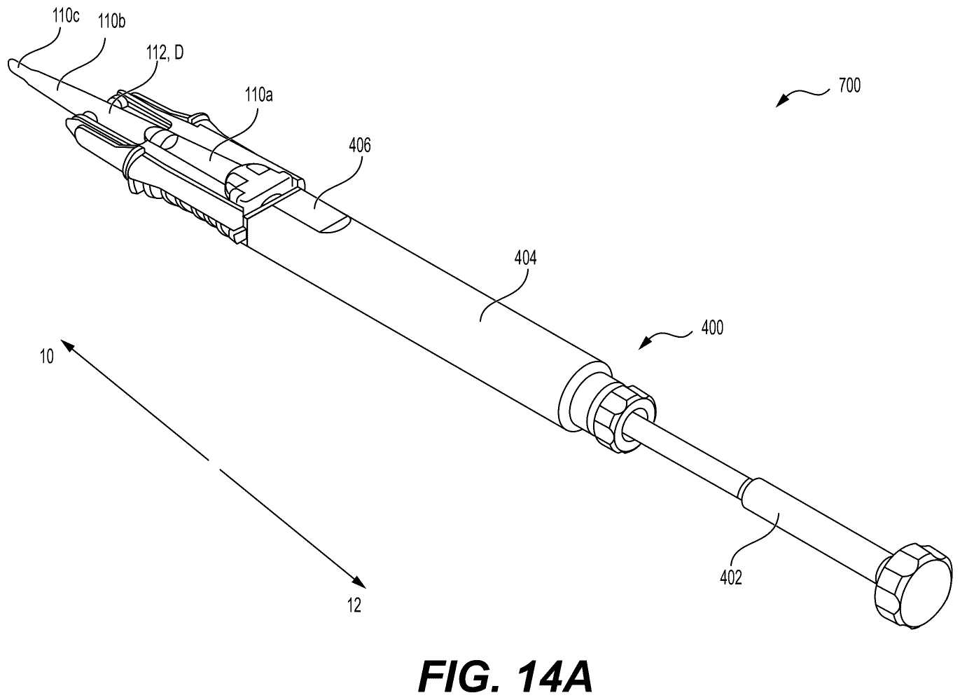

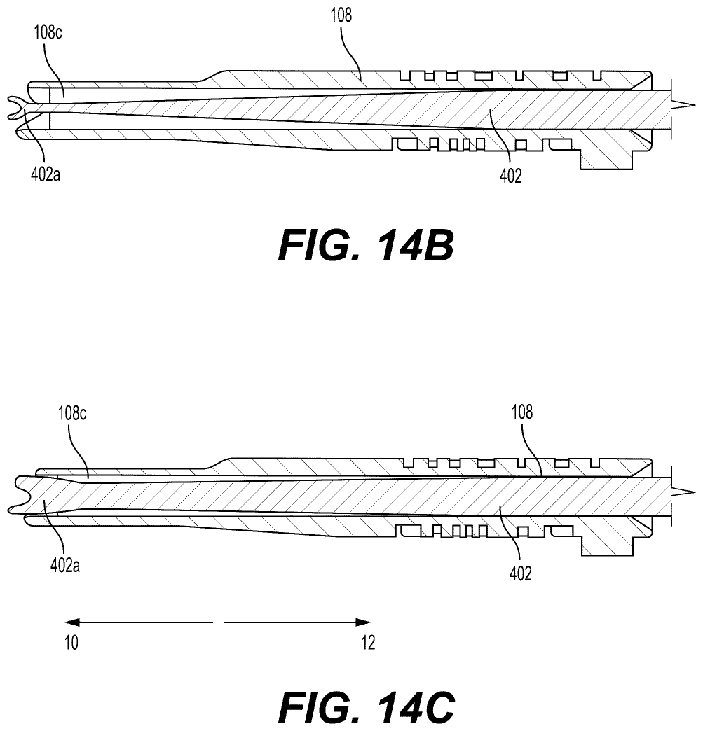

The first tray component 104 can further include a tunnel 308 in which the sleeve 108 can be housed during shipment and/or storage. The tunnel 308 and/or the modular cartridge 110 may include one or more features (e.g., snap features, interference fit) that can interact with complementary components on the sleeve 108 to prevent the sleeve 108 from prematurely advancing distally through the tunnel 108 and/or the modular cartridge 110. For example, as particularly shown in FIGS. 6 and 13A-13B, sleeve 108 may include a protrusion 108b configured to engage with a complementary component (e.g., a recess) along the inner surface of the tunnel 308 to prevent the sleeve 108 from prematurely advancing distally 10 through the tunnel 308. The protrusion 108b can be located along the top, bottom, or side of the sleeve 108, and can be formed into any shape (e.g., non-circular). As the second tray component 106 is pushed into the first tray component 104 (or the first and second tray components 204, 206 pulled away from one another), as discussed herein, enough force must be applied to the second tray component 106 (or the first and/or second tray components 204, 206) such that when it engages with the sleeve 108, the second tray component 106 can force the protrusion 108b out of any complementary feature along the inner surface of the tunnel 308 so that the sleeve 108 can be advanced distally 10 through the tunnel 308 and into the modular cartridge 110. As another example, and as particularly shown in FIG. 13B, once the sleeve 108 is advanced distally 10 through the tunnel 308 and into the modular cartridge 110, the protrusion 108b may engage with a complementary component of the modular cartridge 110, such as recess 111, to prevent the sleeve 108 from moving further distally into the modular cartridge 110. This engagement can also prevent the sleeve 108 from retreating proximally as the modular cartridge 110 is removed and engaged into the delivery device 400 (FIG. 14A).

Turning back to FIG. 4A, the first tray component 104 can include one or more modular cartridge retention devices 318. The modular cartridge retention devices 318 hold the modular cartridge 110 in place during shipping and actuating the second tray component 106. However, the modular cartridge 110 can be removed from the first tray component 104 at any time before or after the actuation of the second tray component 106 without damage to the first tray component 104, the modular cartridge 110, and/or the modular cartridge retention devices 318.

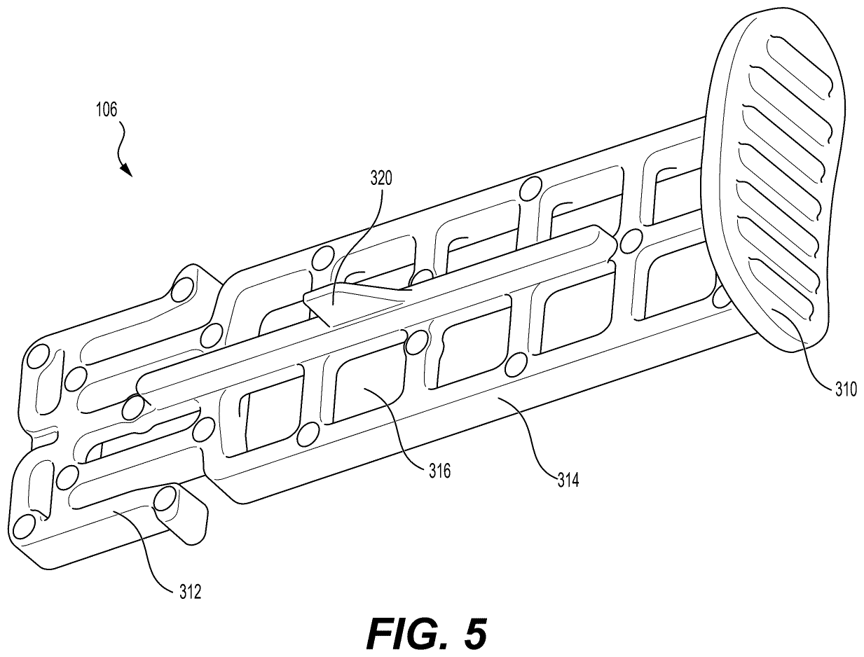

FIG. 5 provides an example second tray component 106 of a lens management device (e.g., 100). As shown, the second tray component 106 can include a handle 310 that can be used, for example, by a physician to push the second tray component 106 distally into the first tray component 104 to advance the sleeve 108 distally into the modular cartridge 110 thereby advancing the lens 112 from its first lens position C to its second lens position D within the modular cartridge 110, as further discussed herein. The second tray component 106 can further include a distal end 312 configured in any size or shape such that it can slide distally 10 into the first tray component 104 to engage with the sleeve 108 to advance the sleeve 108 distally 10 into the modular cartridge 110, as further discussed below. The second tray component 106 can include a central protrusion 320 (also shown in FIG. 1) that engages with the sleeve 108 when the second tray component 106 is pushed into the first tray component 104. That is, the central protrusion 320 can engage with the sleeve 108 to itself push the sleeve 108 distally 10 into the modular cartridge 110. In some embodiments, the tunnel 308 can also be enclosed (e.g., during shipment and/or storage) to further help prevent the sleeve 108 from prematurely advancing into the modular cartridge 110 (e.g., via the central protrusion 320).

As discussed herein, it should be understood that a second tray component 206 (FIG. 2) may include one or more of the same or similar components as the second tray component 106. However, the respective component(s) of second tray component 206 may be configured differently to prevent premature movement of the sleeve 108 before the first and second tray components 204, 206 are pulled away from one another, as discussed above. For example, the second tray component 206 may include one or more protrusions (like central protrusion 320) that engage with one or more complementary components on first tray component 204 to prevent the first and second tray components 204, 206 from prematurely pulling apart from one another thereby prematurely moving the sleeve 108. In some embodiments, these features may also help to prevent premature movement of the first and second tray components 204, 206 and/or the sleeve 108 during shipment and/or storage.

Turning back to FIG. 5, in some embodiments, the second tray component 106 can include a central shaft 314 configured in any size or shape such that it can slide distally 10 into the first tray component 104, as discussed herein. The central shaft 314 may include one or more openings 316 that allow the second tray component 106 to have sufficient rigidity and/or strength while maintaining a desired weight.

FIG. 6A provides an example sleeve 108 of a lens management device (e.g., 100, 200, 300). As shown, the sleeve 108 can include a distal tip 108a configured to engage with the lens 112, as further discussed herein. Additionally, the sleeve 108 can include a lumen 108c therethrough such that a first component 402 (i.e., pushrod) of a delivery device 400 (FIGS. 14A-14C) can be inserted through the sleeve 108 to deliver the lens 112 into a patient's eye, as further discussed below. As discussed above, the sleeve 108 can include a protrusion 108b that engages with one or more complementary components within the tunnel 308 and/or the modular cartridge 110 to prevent the sleeve 108 from prematurely advancing through the tunnel 308 and/or the modular cartridge 110. This protrusion 108b can also help ensure that the sleeve 108 does not rotate and/or translate off the distal and/or proximal lens delivery axis when being advanced so that the features on the distal tip 108a appropriately engage with the lens 112. The sleeve 108 can also include one or more features along its external surface, such as indentations 108d, that can aid in inserting the sleeve 108 through the tunnel 308 and/or modular cartridge 110.

The sleeve 108 can be equipped with one or more features on its distal tip 108a which are designed to facilitate lens contact and folding parameters. For example, the distal tip 108a of the sleeve 108 can have a jaw-like configuration, with a top jaw 108e, bottom jaw 108f, and a space S between the top and bottom jaws 108, 108f. In some embodiments, the top jaw 108e can be designed to contact a haptic. In some embodiments, the top jaw 108e can be designed to manage a haptic as the lens 112 is advanced towards the sleeve 108. In some embodiments, the top jaw 108e can be designed to ensure the trailing haptic folds over the optic portion of a lens 112. In some embodiments, the top jaw 108e can extend in a direction that is perpendicular to the lens traveling direction L to an extent sufficient to facilitate haptic folding over the optic portion of a lens 112. In some embodiments, the top jaw 108e can have a depth (thickness) D1 in a direction that is substantially perpendicular to the lens traveling direction L that is between about 10-40%, or about 10-30%, or about 8-25% of the diameter D2 of the distal tip 108a of the sleeve 108.

In some embodiments, the bottom jaw 108f can be utilized to capture the posterior surface 112b of a lens during advancement (FIG. 13B). The bottom jaw 108f can have a dimension that is the same as the top jaw 108e, or different from the top jaw 108e (e.g., a depth that is less than that of the top jaw 108e). In some embodiments, the distal tip 108a of the sleeve 108 can have one or more cutouts 108g (e.g., 108g-i, 108g-ii) to prevent pinching the haptic optic junction. Cutout 108g-i can be used for haptic-optic junction when the haptic is folded. The one or more cutouts 108g can be lateral cutouts between the top and bottom jaws 108e, 108f.

In some embodiments, the one or more cutouts 108g may include at least two separate cutouts, 108g-i and 108g-ii, that have respectively varying dimensions. For example, cutout 108g-ii may be shallower than cutout 108g-i. The length of the top jaw 108e can be varied in relation to a tangent line along the inside of the cutout 108g-ii to aid in capturing the lens 112 more efficiently and/or effectively. For example, if a depth of the cutout 108g-ii is too deep, and hence the extended length of the top jaw 108e too long, this configuration could make capturing the lens 112 more challenging. Configuring the cutout 108g-ii with a shorter depth may allow the lens 112 to float slightly, allowing for improved capturing of the lens 112 by the sleeve 108. In some embodiments, the cutout 108g-i may be configured to have a smooth inner curvature and/or surface to aid in reducing point pressure that may be created by a more angular/elbow configuration (as particularly shown in FIG. 6A).

FIGS. 6B and 6C provide examples of alternative distal tips that may be used with a sleeve 108. As shown, these alternative distal tips can still provide the sleeve with a lumen 108c-1 (6B) or 108c-2 (6C) therethrough. As shown, these distal tips can also have varied dimensions and/or shapes of their respective features compared to distal tip 108a (FIG. 6A).

As shown in FIG. 6B, distal tip 108a-1 can have a reduced top jaw 108e-1 that allows space for larger lenses to float and/or ensures efficient capture of the optic edge. It can also have a bottom jaw 108f-1 that includes a smooth surface to reduce compression of the sleeve on a posterior surface of the lens, as well as a space S-1 between the top and bottom jaws 108e-1, 108f-1. Distal tip 108a-1 can also have one or more cutouts 108g-i-1, 108g-ii-1 that can each have a unique size and/or shape, and/or can be different sizes and/or shapes compared to cutouts 108g-i and 108g-ii of distal tip 108a (6A).

As shown in FIG. 6C, distal tip 108a-2 can have a longer top jaw 108e-2 that extends past the tangency of the joining feature between the top and bottom jaws 108e-2, 108f-2 to aid in maintenance of the trailing haptic in a forward folded position. Distal tip 108a-2 can have a space S-2 between its top and bottom jaws 108e-2, 108f-2. The bottom jaw 108f-2 can also have a reduction of its haptic-optic junction opening to reduce lens compression when folded in this component. The reduction in compression can reduce a risk of cosmetic artifacts that are a result of compression of the lens and the haptic. This feature can have a diameter D3 that is larger in diameter than the diameter D4 of the opposite mouth. Some clearance is still required to allow for the haptic-optic junction of the lens to sit during lens advancement.

As particularly shown in FIG. 13B, in the embodiment of the invention where the top jaw 108e engages the haptic 112a, and the bottom jaw 108f captures the posterior surface 112b of a lens 112, a portion of the periphery of the optical portion of the lens 112 sits within the opening S created between the top and bottom jaws 108, 108f.

FIG. 7 shows a top perspective view of a modular cartridge 110 that includes a lens module 110a and a cartridge 110b. The modular cartridge 110 can also include a distal end or tip 110c (e.g., as part of the cartridge 110b), as further discussed below.

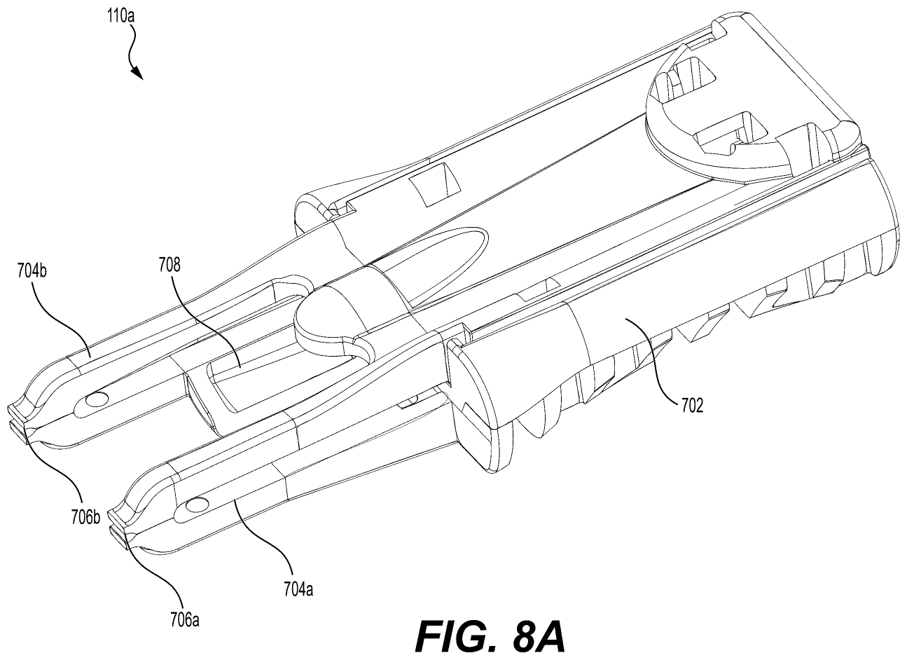

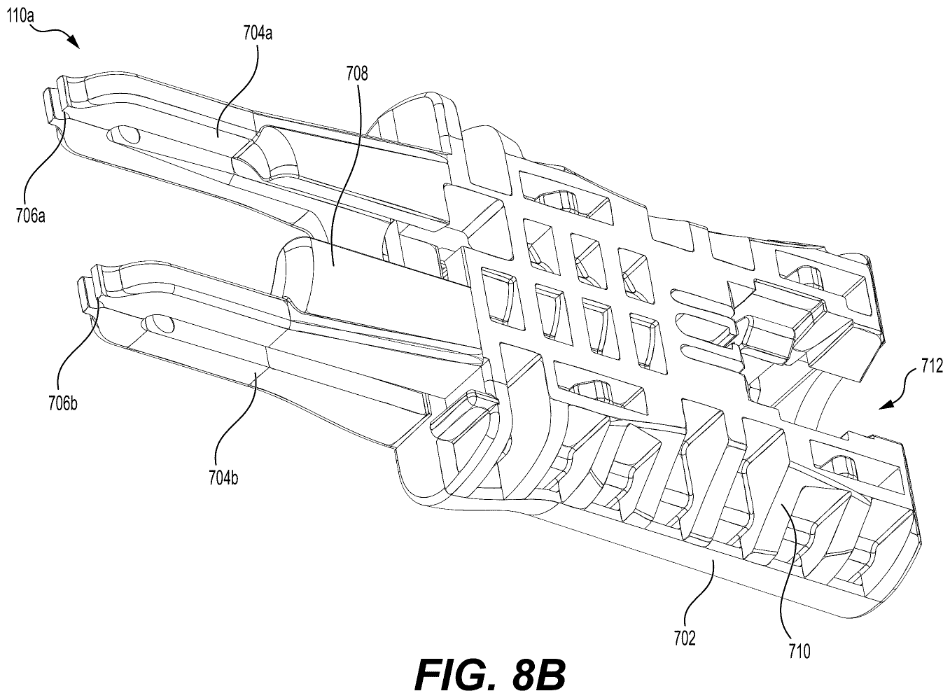

FIGS. 8A and 8B respectively provide top and bottom perspective views of a lens module 110a of a modular cartridge 110. As shown, the lens module 110a can include a body 702, and one or more extensions 704a, 704b each having a respective hinge 706a, 706b. Lens module 110a can further include a tab 708 configured to engage with a cartridge 110b of the lens module 110a, as further discussed below. As particularly shown in FIG. 8B, the lens module 110a can include one or more ribs 710 to provide stability and strength, as well as an opening 712 to engage with a delivery device, such as delivery device 400, as further discussed below (FIG. 14A).

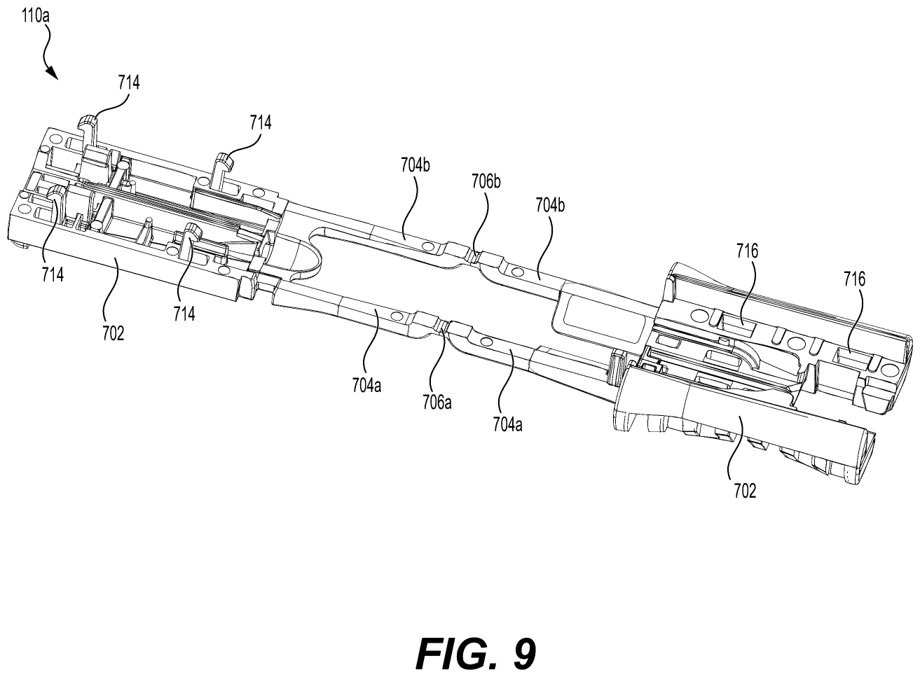

FIG. 9 provides an illustration of the lens module 110a when it is opened or extended via the hinges 706a, 706b. The hinges 706a. 706b can be separate structures or formed as living hinges. Lens module 110a may be opened or extended in such fashion during manufacturing and/or assembly. During the assembly of some embodiments, a lens 112 can be placed into the lens module 110a unfolded and in its at-rest position and then transported in that condition to the user. Additionally, in some embodiments, a lens 112 may be packaged in a separate enclosure for storage within the lens module 110a during shipment. Once the lens module 110a is shipped, a user, e.g., a physician, may open the lens module 110a, as shown, and remove the lens 112 from its packaging held within the lens module 110a such that the lens 112 can be implanted in cartridge 110b of the modular cartridge 110 to prepare for handling and delivery into a patient's eye, as discussed herein. In some embodiments, lens module 110a may further include one or more retention devices 714, such as on one side of the lens module 110a, that may engage with one or more complementary components, such as recesses 716, on the opposite side. The retention device(s) 714 and recess(es) 716 may engage with one another such that the lens module 110a may be closed and seated in place, as shown in FIGS. 8A and 8B.

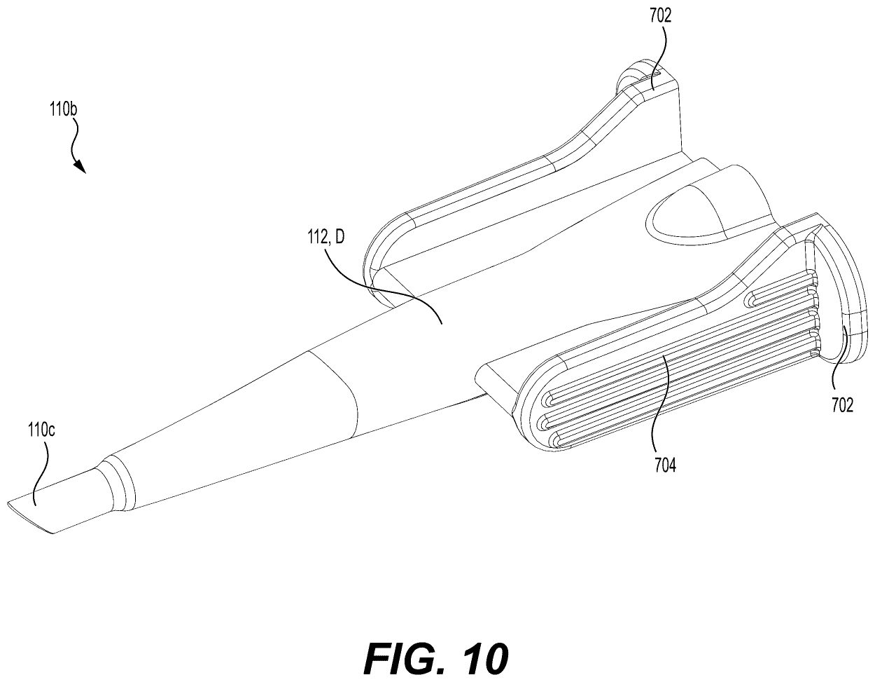

FIG. 10 provides a top perspective view of a cartridge 110b of a modular cartridge 110. As shown, the cartridge 110b can include a distal end or tip 110c through which the lens 112 is delivered into the patient's eye, as further discussed below. The cartridge 110b can include one or more side rails 704 and/or one or more extensions 702 to provide rigidity and strength to the cartridge 110b, as well as to aid in engaging the cartridge 110b with the lens module 110a, as further discussed below.

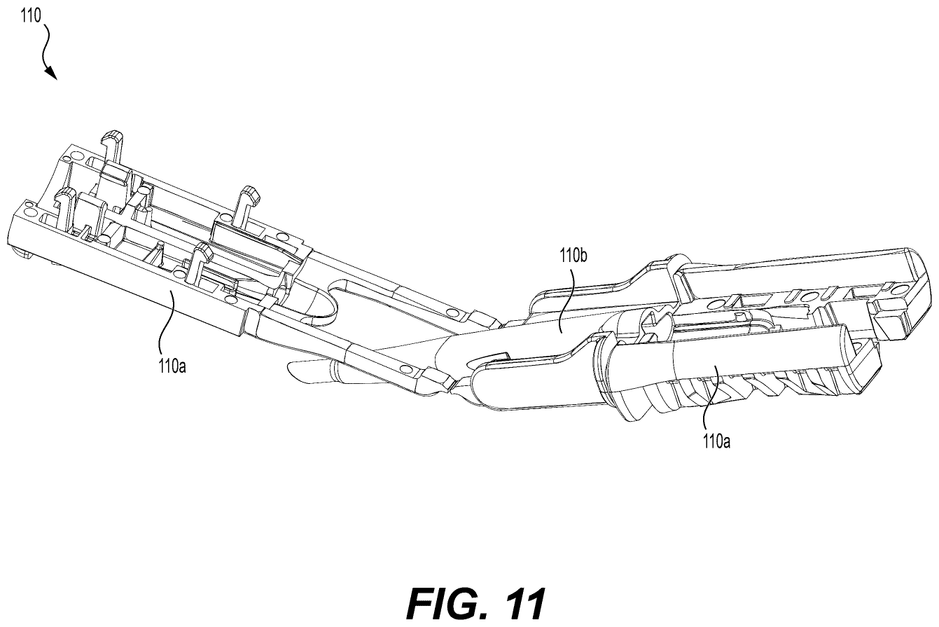

FIG. 11 illustrates how the lens module 110a and cartridge 110b can be engaged to form the modular cartridge 110. As shown (and discussed above), in some embodiments, the lens module 110a can be extended into its open position (e.g., via hinges 706a, 706b). The cartridge 110b can then be engaged with the lens module 110a via the extension(s) 702, and the lens module 110a closed and seated, as discussed above (FIG. 9), to sandwich the cartridge 110b between the two sides of the lens module 110a (FIG. 7). This process may be conducted during manufacturing of the modular cartridge 110 such that the lens module 110a and cartridge 110b are already engaged when put in use by a user, e.g., a physician.

FIG. 12 provides a top view of the modular cartridge 110 housed within the first tray component of a lens management device (e.g., first tray component 104 of device 100). As shown, the lens 112 can first be situated in a first lens position C within the modular cartridge 110. This first lens position C can be used during shipment and/or storage of the lens management device. Additionally, this first lens position C, or a holding position, allows the lens 112 to be hydrated (e.g., with a balanced salt solution) prior to the lens 112 being advanced distally 10 within the modular cartridge 110. This first lens position C within the modular cartridge 110 can also allow the lens 112 to begin the elongation process necessary to deliver the lens 112 through a minimal sized distal end 110c of the modular cartridge 110. With respect to the lens management device 100 of FIG. 1, once the physician is ready to advance the lens 112 to its second lens position D to prepare the lens 112 for delivery into the eye, the second tray component 106 of the tray 102 can be pushed distally 10 from its first position A (FIG. 1) to a second position whereby the second tray component 106 can be telescoped within the first tray component 104, as discussed herein. With respect to the lens management device 200 of FIG. 2, to advance the lens 112 to its second position D, the first and second tray components 204, 206, starting in their first position E (FIG. 2), can be pulled away from one another (or one component pulled away from the other component while the other component is held stationary) to transition the tray 202 to its second position thereby sliding the modular cartridge 110 proximally 12 to engage the modular cartridge 110 with the distal tip 108a of the sleeve 108. With either lens management device (100 or 200), as the lens 112 is transitioned from its first lens position C to its second lens position D, the lens 112 can be at least partially folded.

There also can be internal features of the lens module 110a which facilitate lens folding. Some of these features include protrusions, ramps, or lips which encourage curving of the lens as it advances past them; other, different protrusions, ramps, or lips similarly encourage folding of the haptics over the optic as the lens 112 advances.

FIGS. 13A and 13B provide cross-sectional views of the sleeve 108 being advanced distally 10 into the modular cartridge 110.

As shown in FIG. 13A, the distal tip 108a of the sleeve 108 has not yet engaged with the lens 112 when the lens 112 is in its first lens position C within the modular cartridge 110. As shown in FIG. 13B, when the sleeve 108 is advanced distally 10 within the modular cartridge 110 (either via the second tray component 106 being pushed distally 10 into the first tray component 104, or via the first and second tray components 204, 206 being pulled apart in relation to one another), the distal tip 108a of the sleeve 108 engages with the lens 112 to advance the lens 112 distally 10 to its second lens position D within the modular cartridge 110. The distal tip 108a of the sleeve 108 can be configured to have a shape that promotes folding of the trailing haptic of the lens 112 onto the anterior surface of the body of the lens 112. The distal tip 108a can also be shaped to consistently advance lenses of varying geometries from the storage or first lens position C to the holding or second lens position D. The shape of the sleeve 108 and/or the distal tip 108a can also be configured such that the sleeve 108 can maintain the lens 112 on the same axis as the lens 112 is advanced from the first lens position C to the more distal second lens position D within the modular cartridge 110, which promotes folding of the edges of the lens 112 up and over the optic. This step can promote folding of at least one haptic over the optic portion of the lens 112. The edges of the optic portion of the lens 112 can be raised so that the lens 112 is no longer in a planar configuration and/or so that the edges of the optic portion extend over at least a portion of the haptics.

As particularly shown in FIG. 13B, once the lens 112 is advanced from the first lens position C to the more distal second lens position D within the modular cartridge 110, the sleeve 108 can be fully encased in the modular cartridge 110. Further, forward advancement of the sleeve 108 into the modular cartridge 110 can be stopped due to an interference fit with the inner diameter of the modular cartridge 110 once the lens 112 is in its holding or second lens position D. The degree of advancement of the lens 112 can be controlled by a length L1 of the sleeve 108 (FIG. 6), and one of ordinary skill in the art will be able to determine how far the lens 112 should be advanced in this first step, and select an appropriate length L1 of the sleeve 108, and internal features of the cartridge 110b and lens module 110a, to facilitate a desired degree of folding of the lens 112.

Once the lens 112 is advanced from its first lens position C to its second lens position D, and the sleeve 108 encased in the modular cartridge 110 (and the tray 102 or 202 in its second position), the modular cartridge 110, sleeve 108, and lens 112 can be disengaged from the tray 102 (or 202). That is, the first and second tray components 104, 106 (or 204, 206) can be snapped off from the modular cartridge 110. Additionally, or alternatively, the tray 102 (or 202) can be configured such that it permits the release of the modular cartridge 110 when the tray 102 (or 202) is in its first position A (or E).

FIG. 14A shows an example lens management system 700 after the modular cartridge 110, with the sleeve 108 and lens 112 encased therein, are removed from the tray 102 (or 202). At this stage of the lens management process, a delivery device 400 can be engaged with the modular cartridge 110. The delivery device 400 can include a first component 402, such as a plunger and/or pushrod, and a second component 404, such as a barrel. The second component 404 can be disposable or reusable, or reusable in part. A distal end 406 of the delivery device 400 can be shaped accordingly to engage with the shape of the modular cartridge 110. The first component 402 (for example, the pushrod) of the delivery device 400 can be advanced distally 10 into the second component 404 (e.g., similar to pushing a plunger into the barrel of a syringe or by a threaded engagement, or a combination of both).

FIGS. 14B-14C show examples of a first component 402 (pushrod) as it is advanced distally through the sleeve 108. The first component 402 (i.e. pushrod) can be advanced distally 10 through the lumen 108c of the sleeve (at this point encased within the modular cartridge 110) such that a distal tip 402a of the first component 402 (pushrod) can engage with the lens 112 in its second lens position D (FIG. 13B). The distal tip 402a of the first component 402 (pushrod) can be made of a non-rigid material to reduce potential abrasion of the lens 112 during delivery. The distal tip 402a can also be made of different materials and/or be of different shapes (as particularly shown in FIGS. 14B and 14C). Additionally, as the first component 402 (pushrod) should be small enough to accommodate a small incision size in a patient's eye, the sleeve 108 provides additional structural support within the modular cartridge 110 to prevent bending or flexing of the first component 402 (pushrod) throughout the full length of advancement of the pushrod, providing support and stability through the entire lens delivery process. Such bending or flexing may be seen with lenses which require more force to advance. Having the sleeve 108 present in the device (e.g., 100), and allowing the first component 402 to advance through the sleeve 108, also provides the insertion system manufacturer with the opportunity to select materials that are not as stiff but may offer other advantages.

The force applied to the first component 402 (e.g., by a physician) is transferred to the lens 112 to advance the lens 112 from its second lens position D distally 10 to and through the distal end 110c of the modular cartridge 110 and into a patient's eye, as particularly shown in FIGS. 13A-13B. As the lens 112 is advanced through the distal end or tip 110c of the modular cartridge, the lens 112 can be further folded. The lens 112 can also stretch along the distal and/or proximal lens delivery axis as the lens 112 is being advanced through the distal end 110c (e.g., nozzle) for placement in the patient's eye.

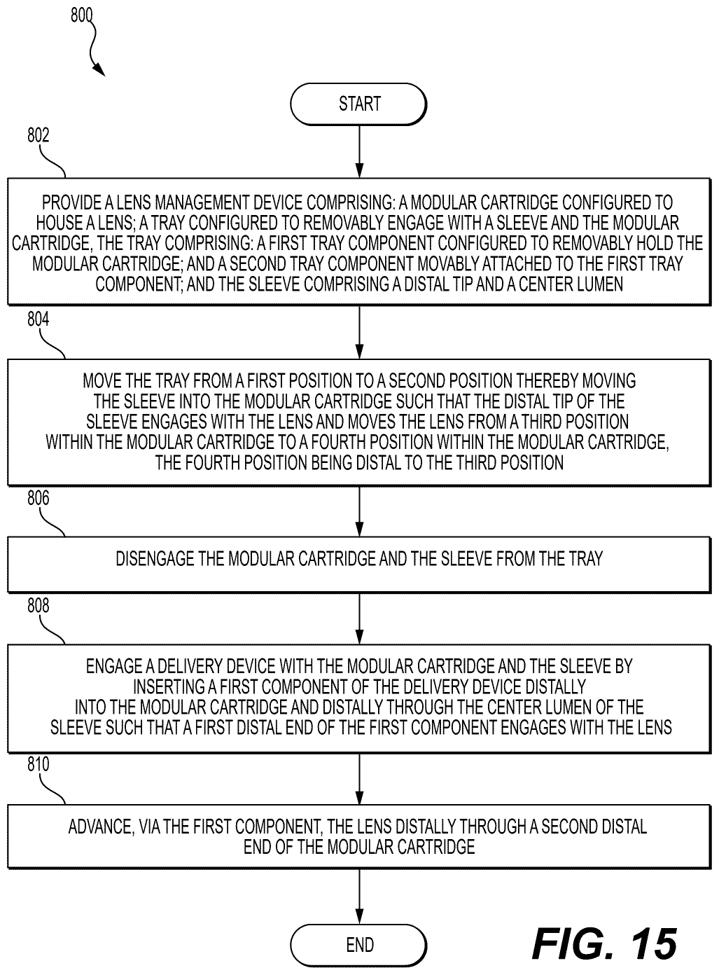

FIG. 15 provides a flowchart of an example method 800 for lens management. In block 802, the method 800 can include providing a lens management device, such as lens management device 100 or 200. As discussed herein, the device can include a tray (e.g., tray 102 or 202) including first and second tray components (e.g., 104, 106 or 204, 206). The tray can be configured to removably engage with a sleeve (e.g., sleeve 108) and a modular cartridge (e.g., modular cartridge 110). The first tray component can be configured to removably hold the modular cartridge, while the second tray component can be movably attached to the first tray component, as discussed herein.

In block 804, the method 800 can include moving the tray from a first position to a second position thereby moving the sleeve into the modular cartridge such that the distal tip of the sleeve engages with the lens and moves the lens from a third position within the modular cartridge to a fourth position within the modular cartridge, the fourth position being distal to the third position. At this step, the lens can be at least partially folded as it transitions from the third to the fourth position.

In block 806, the method 800 can include disengaging the modular cartridge and the sleeve from the tray. In this step, the lens and sleeve may be encased within the modular cartridge, as discussed above, such that all three components can be removed from the tray.

In block 808, the method 800 can include engaging a delivery device (e.g., delivery device 400) with the modular cartridge and the sleeve. This step can include inserting a first component (e.g., first component 402) of the delivery device distally into the modular cartridge and distally through a center lumen of the sleeve such that a distal end of the first component engages with the lens encased therein.

In block 810, the method 800 can include advancing, for example, via the first component, the lens distally through a distal end of the modular cartridge such that the lens can be delivered into a patient's eye. At this step, the lens can be further folded as it is delivered through the distal end of the modular cartridge.

In some examples, disclosed systems or methods may involve one or more of the following clauses:
Clause 1: A lens management device comprising: a modular cartridge configured to house a lens; a tray configured to transition from a first position to a second position and comprising: a first tray component configured to removably hold the modular cartridge; and a second tray component movably attached to the first tray component; and a sleeve, comprising a distal tip and a center lumen, configured to move distally into the modular cartridge as the tray transitions from the first position to the second position, wherein the modular cartridge is configured to house the lens in a third position when the tray is in the first position, and in a fourth position when the tray is in the second position, the fourth position being distal to the third position, and wherein transitioning the tray from the first position to the second position causes the sleeve to enter into the modular cartridge such that the distal tip of the sleeve is positioned to engage with the lens and move the lens from the third position to the fourth position.
Clause 2: The lens management device of clause 1, wherein the second tray component is movable from the first position to the second position.
Clause 3: The lens management device of clause 2, wherein transitioning the tray from the first position to the second position comprises moving the second tray component distally into the first tray component.
Clause 4: The lens management device of clause 1, wherein one or more of the first tray component and the second tray component is movable from the first position to the second position.
Clause 5: The lens management device of clause 1, wherein the distal tip comprises: a top jaw; a bottom jaw; a first cutout disposed at a first junction between the top and bottom jaws, the first cutout having a first diameter; and a second cutout disposed at a second junction between the top and bottom jaws, the second cutout having a second diameter, wherein the first diameter is greater than the second diameter.
Clause 6: The lens management device of clause 1, wherein the distal tip is configured to reduce compression of the lens when the sleeve engages with the lens and moves the lens from the third position to the fourth position.
Clause 7: The lens management device of clause 1, wherein the tray is configured to permit the release of the modular cartridge in the second position.
Clause 8: The lens management device of clause 1, further comprising a lens housed in the modular cartridge.
Clause 9: The lens management device of clause 8, wherein the lens comprises an intraocular lens (IOL).
Clause 10: A lens management device comprising: a tray configured to transition from a first position to a second position and comprising: a first tray component configured to hold a lens; and a second tray component movably attached to the first tray component; and a sleeve, comprising a distal tip and a center lumen, configured to move distally into the first tray component as the tray transitions from the first position to the second position, wherein transitioning the tray from the first position to the second position moves the sleeve into the first tray component such that the distal tip of the sleeve is positioned to engage with the lens and move the lens from a third position within the first tray component to a fourth position within the first tray component, the fourth position being distal to the third position.
Clause 11: The lens management device of clause 10, wherein the second tray component is movable from the first position to the second position, and wherein transitioning the tray from the first position to the second position comprises moving the second tray component distally into the first tray component.
Clause 12: The lens management device of clause 10, wherein the second tray component is movable from the first position to the second position, and wherein transitioning the tray from the first position to the second position comprises one or more of moving the second tray component proximally in relation to the first tray component, and moving the first tray component distally in relation to the second tray component.
Clause 13: The lens management device of clause 10, further comprising a lens housed in the first tray component.
Clause 14: A method for managing a lens, the method comprising: providing a lens management device comprising: a modular cartridge configured to house the lens; a tray configured to removably engage with a sleeve and the modular cartridge, the tray comprising: a first tray component configured to removably hold the modular cartridge; and a second tray component movably attached to the first tray component; and the sleeve comprising a distal tip and a center lumen; and moving the tray from a first position to a second position thereby moving the sleeve into the modular cartridge such that the distal tip of the sleeve engages with the lens and moves the lens from a third position within the modular cartridge to a fourth position within the modular cartridge, the fourth position being distal to the third position.
Clause 15: The method of clause 14, wherein moving the tray from the first position to the second position comprises moving the second tray component distally into the first tray component.
Clause 16: The method of clause 14, wherein moving the tray from the first position to the second position comprises one or more of moving the first tray component distally in relation to the second tray component, and moving the second tray component proximally in relation to the first tray component.
Clause 17: The method of clause 14, further comprising: disengaging the modular cartridge and the sleeve from the tray; engaging a delivery device with the modular cartridge and the sleeve by inserting a first component of the delivery device distally into the modular cartridge and distally through the center lumen of the sleeve such that a first distal end of the first component engages with the lens; and advancing, via the first component, the lens distally through a second distal end of the modular cartridge.
Clause 18: The method of clause 17, wherein the movement of the lens from the third position to the fourth position partially folds the lens.
Clause 19: The method of clause 18, wherein the advancing step further folds the lens for delivery through the second distal end of the modular cartridge.
Clause 20: The method of clause 14, wherein the lens comprises an intraocular lens (IOL).

The descriptions contained herein are examples of embodiments of the invention and are not intended in any way to limit the scope of the invention. As described herein, the invention contemplates many variations and modifications of structures and methods, including alternative materials, alternative configurations of component parts, and alternative method steps. Modifications and variations apparent to those having skill in the pertinent art according to the teachings of this disclosure are intended to be within the scope of the claims which follow.

What is claimed is:

1. A lens management device comprising:
a modular cartridge configured to house a lens;
a tray configured to transition from a first position to a second position and comprising:
a first tray component configured to removably hold the modular cartridge; and
a second tray component movably attached to the first tray component; and
a sleeve, comprising a distal tip and a center lumen, configured to move distally into the modular cartridge as the tray transitions from the first position to the second position,
wherein the modular cartridge is configured to house the lens in a third position when the tray is in the first position, and in a fourth position when the tray is in the second position, the fourth position being distal to the third position, and
wherein transitioning the tray from the first position to the second position causes the sleeve to enter into the modular cartridge such that the distal tip of the sleeve is positioned to engage with the lens and move the lens from the third position to the fourth position.

2. The lens management device of claim 1, wherein the second tray component is movable from the first position to the second position.

3. The lens management device of claim 2, wherein transitioning the tray from the first position to the second position comprises moving the second tray component distally into the first tray component.

4. The lens management device of claim 1, wherein one or more of the first tray component and the second tray component is movable from the first position to the second position.

5. The lens management device of claim 1, wherein the distal tip comprises:
a top jaw;
a bottom jaw;
a first cutout disposed at a first junction between the top and bottom jaws, the first cutout having a first diameter; and
a second cutout disposed at a second junction between the top and bottom jaws, the second cutout having a second diameter,
wherein the first diameter is greater than the second diameter.

6. The lens management device of claim 1, wherein the distal tip is configured so that there is a space between an internal wall of the sleeve and the lens when the sleeve engages with the lens and moves the lens from the third position to the fourth position.

7. The lens management device of claim 1, wherein the tray is configured to permit the release of the modular cartridge in the second position.

8. The lens management device of claim 1, further comprising a lens housed in the modular cartridge.

9. The lens management device of claim 8, wherein the lens comprises an intraocular lens (IOL).

10. A lens management device comprising:
a tray configured to transition from a first position to a second position and comprising:
a first tray component configured to hold a lens; and
a second tray component movably attached to the first tray component; and
a sleeve, comprising a distal tip and a center lumen, configured to move distally into the first tray component as the tray transitions from the first position to the second position,
wherein transitioning the tray from the first position to the second position moves the sleeve into the first tray component such that the distal tip of the sleeve is positioned to engage with the lens and move the lens from a third position within the first tray component to a fourth position within the first tray component, the fourth position being distal to the third position.

11. The lens management device of claim 10, wherein the second tray component is movable from the first position to the second position, and wherein transitioning the tray from the first position to the second position comprises moving the second tray component distally into the first tray component.

12. The lens management device of claim 10, wherein the second tray component is movable from the first position to the second position, and wherein transitioning the tray from the first position to the second position comprises one or more of moving the second tray component proximally in relation to the first tray component, and moving the first tray component distally in relation to the second tray component.

13. The lens management device of claim 10, further comprising a lens housed in the first tray component.

14. A method for managing a lens, the method comprising:
providing a lens management device comprising:
a modular cartridge configured to house the lens;
a tray configured to removably engage with a sleeve and the modular cartridge, the tray comprising:
a first tray component configured to removably hold the modular cartridge; and
a second tray component movably attached to the first tray component; and
the sleeve comprising a distal tip and a center lumen; and
moving the tray from a first position to a second position thereby moving the sleeve into the modular cartridge such that the distal tip of the sleeve engages with the lens and moves the lens from a third position within the modular cartridge to a fourth position within the modular cartridge, the fourth position being distal to the third position.

15. The method of claim 14, wherein moving the tray from the first position to the second position comprises moving the second tray component distally into the first tray component.

16. The method of claim 14, wherein moving the tray from the first position to the second position comprises one or more of moving the first tray component distally in relation to the second tray component, and moving the second tray component proximally in relation to the first tray component.

17. The method of claim 14, further comprising:
disengaging the modular cartridge and the sleeve from the tray;
engaging a delivery device with the modular cartridge and the sleeve by inserting a first component of the delivery device distally into the modular cartridge and distally through the center lumen of the sleeve such that a first distal end of the first component engages with the lens; and
advancing, via the first component, the lens distally through a second distal end of the modular cartridge.

18. The method of claim 17, wherein the movement of the lens from the third position to the fourth position partially folds the lens.

19. The method of claim 18, wherein the advancing step further folds the lens for delivery through the second distal end of the modular cartridge.

20. The method of claim 14, wherein the lens comprises an intraocular lens (IOL).
